(19) United States
(12) Patent Application Publication
(10) Pub. No.: US 2012/0082631 A1
(43) Pub. Date: Apr. 5, 2012

(54) COMPOSITIONS

(76) Inventors: David Jonathan Bradshaw, Ashford Kent (GB); Paula Maria Cawkill, Ashford Kent (GB); Roy Downey, Lympne Kent (GB); Linda Kehaya, Ashford Kent (GB); John Martin Behan, Ashford Kent (GB)

(21) Appl. No.: 13/266,995

(22) PCT Filed: May 10, 2010
(86) PCT No.: PCT/EP2010/056329

§ 371 Date: Dec. 8, 2011

(30) Foreign Application Priority Data
May 8, 2009 (GB) 0907925.2

(51) Int. Cl. A61K 8/36; A61Q 11/00; A61K 8/27; A61K 8/19

(52) U.S. Cl. 424/55; 424/49

(57) ABSTRACT

An oral care composition comprising at least one polyvalent metal ion salt and at least one of maltol (M), ethyl maltol (E), and cyclotene (C) such that the concentration (in ppm by weight of the total oral care composition) of maltol (M), ethyl maltol (E), and cyclotene (C) meet the criterion (M/50+E/50+C/250)≧1. The compositions have reduced astringency.

### FIELD OF THE INVENTION

This invention relates to a method of reducing the astringency of an oral care composition, to reduced astringency oral care compositions, and to the use of certain compounds to reduce astringency in oral care compositions.

### BACKGROUND TO THE INVENTION

For many years polyvalent metal (PVM) ion salts, particularly those of zinc, have been included in oral care compositions for their beneficial properties and effects, examples of which include antimicrobial, anti-plaque, anti-tartar and malodour control and/or prevention. However, unfortunately, the use of PVM ion salts is often associated with adverse effects, in particular astringency.

Astringency, in respect to oral compositions, is defined as a complex group of sensations experienced when a substance causes the oral surfaces to feel rough, the mouth to feel dry, and the mucosal surfaces to tighten, draw or pucker.

The problem of astringency associated with PVM ions salts, particularly those of zinc, has been known for some time, and considerable effort has been expended in seeking solutions to it.

U.S. Pat. No. 4,416,867 teaches that under certain pH conditions the addition of glycine to zinc-containing toothpaste compositions ameliorates astringency.

US Patent Application Publication No. 2008/0138298 discloses oral care compositions comprising 0.01-10% of polyphosphorylated inositol derivatives such as phytic acid. These compositions are said to be effective in preventing/controlling disease states whilst having improved aesthetics such as astringency.

PVM ion salt astringency has been addressed using monomenthyl succinate salts (WO 98/11867) and poloxamer polymers (WO 00/32160).

However, the methods known in the art for combating astringency caused by PVM ion salts have not been entirely successful.

The applicant has now found that, by the addition to oral care compositions of certain known flavour ingredients that are readily available, relatively inexpensive, and GRAS approved, the perception of PVM ion salt induced astringency may be reduced.

### SUMMARY OF THE INVENTION

In a first aspect of the present invention there is provided a method of reducing the astringency of an oral care composition containing PVM ion salts, the method comprising the addition to an oral care composition of at least one of maltol (3-hydroxy-2-methyl-4H-pyran-4-one), ethyl maltol (2-ethyl-3-hydroxy-4H-pyran-4-one), and cyclotene (2-hydroxy-3-methyl-2-cyclopenten-1-one); such that the concentrations of maltol, ethyl maltol, and cyclotene in said oral care composition meet the criterion (M/50+E/50+C/250)≧1, particularly 1.0-1.5, more particularly 1.2-1.5; wherein M, E and C represent the concentration (in ppm by weight of the total oral care composition) of maltol, ethyl maltol, and cyclotene respectively.

The term “oral care composition” as used herein refers to non-food compositions that are designed to be taken into the mouth to deliver a variety of benefits. Such compositions include dentifrices, mouthwashes, mouth sprays and gargle compositions, breath strips (edible films placed in the oral cavity to administer thereto an active agent such as a flavourant or breath-freshening agent), and chewing gums. The term “dentifrice”, as used herein, means toothpaste, oral care gels or liquids, unless otherwise specified. The dentifrice composition may be a single-phase composition or it may be a combination of two or more separate dentifrice compositions. The dentifrice composition may be in any desired form, such as deep striped, surface striped, multilayered, having the gel surrounding the paste, or any combination thereof.

In another aspect of the present invention, there is provided an oral care composition comprising at least one PVM ion salt, in particular a zinc salt, and an astringency-counteracting amount of at least one of maltol, ethyl maltol, and cyclotene, such that the concentrations in the said oral care composition of maltol, ethyl maltol, and cyclotene meet the criterion (M/50+E/50+C/250)≧1, particularly 1.0-1.5, more particularly 1.2-1.5; wherein M, E and C represent the concentration (in ppm by weight of the total oral care composition) of maltol, ethyl maltol, and cyclotene respectively.

In a further aspect of the present invention there are provided compositions comprising at least one of maltol, ethyl maltol, and cyclotene, for use in the reduction of the astringency of an oral care composition comprising a PVM ion salt, the concentrations of maltol, ethyl maltol, and cyclotene in the oral care composition being such that they meet the criterion (M/50+E/50+C/250)≧1, wherein M, E and C represent the concentration (in ppm by weight of the total oral care composition) of maltol, ethyl maltol, and cyclotene respectively.

Maltol, ethyl maltol, and cyclotene may be used alone or in combination as the sole components in a composition. Alternatively they may be employed in conjunction with one or more other ingredients commonly used in the art.

In yet another aspect of the present invention, there is provided a method of forming an oral care composition, the method comprising the addition to an oral care composition of at least one of maltol (3-hydroxy-2-methyl-4H-pyran-4-one), ethyl maltol (2-ethyl-3-hydroxy-4H-pyran-4-one), and cyclotene (2-hydroxy-3-methyl-2-cyclopenten-1-one); such that the concentrations in the oral care composition of maltol, ethyl maltol, and cyclotene meet the criterion (M/50+E/50+C/250)≧1, particularly 1.0-1.5, more particularly 1.2-1.5; wherein M, E and C represent the concentration (in ppm by weight of the total oral care composition) of maltol, ethyl 15 maltol, and cyclotene respectively.

Maltol, ethyl maltol, and cyclotene , or compositions containing one or more said compounds can be added to oral care compositions by using conventional techniques to directly admix the said compounds, or composition into the oral care composition.

Subject to the proviso that (M/50+E/50+C/250)≧1, the concentrations of maltol, ethyl maltol, and cyclotene that may be employed in the abovementioned oral care compositions will depend on the concentration of PVM ion salts in the oral care composition. It will also depend on the particular sensorial effect that the formulator is trying to achieve, as these materials are flavoured and, depending on the nature of other flavour ingredients present, may influence the overall hedonic or sensorial effect of the oral care composition.

Having regard to these considerations, and the teaching herein, the skilled person will be able, by routine experimentation, to find the appropriate concentration for each ingredient employed.

In a particular embodiment maltol may be employed at levels of at least 50 ppm, more particularly 100 ppm, still more particularly 250 ppm.

In a particular embodiment, ethyl maltol may be employed at levels of at least 50 ppm, more particularly 100 ppm, still more particularly 250 ppm.

In a particular embodiment, cyclotene may be employed at levels of at least 250 ppm, more particularly 500 ppm, still more particularly 750 ppm.

In a more particular embodiment, cyclotene is combined with one or both of maltol and ethyl maltol at levels mentioned hereinabove.

In a further particular embodiment, cyclotene is combined with one or both of maltol and ethyl maltol in a ratio of 1:10 to 10:1, more particularly 1:5 to 5:1.

As mentioned herein, PVM ion salts employed in oral care products can cause astringency. Typical PVM ion salts used in oral care compositions of the present invention are zinc salts, particularly zinc chloride, zinc citrate, zinc acetate, and zinc sulphate, more particularly zinc citrate.

The proportion of PVM ion salts usually employed is 0.1 to 2% by weight of the total oral care composition.

The reduced astringency oral care compositions of the present invention may also comprise one or more additional ingredients or excipients conventionally used in conjunction with oral 20 care compositions for example, additional flavour compounds and other auxilliary agents commonly used in the art.

Examples of known additional flavour ingredients may be found in one of the FEMA (Flavour and Extracts Manufacturers Association of the United States) publications or a compilation thereof which is available from and published by FEMA and contains all FEMA GRAS (Generally Regarded As Safe) publications from 1965 to present, eg GRAS 21 published 2003, or in Allured's Flavor and Fragrance Materials 2004, published by Allured Publishing Inc. Examples of known excipients for oral care products may be found in Gaffar, Abdul, Advanced Technology, Corporate Technology, Department of Oral Care, Colgate-Palmolive Company, Piscataway, N.J., USA. Editor(s): Barel, Andre O.; Paye, Marc; Maibach, Howard I., Handbook of Cosmetic Science and Technology (2001), p. 619-643. Publisher: Marcel Dekker, Inc., New York, N.Y., and in Cosmetics: Science and technology, 2nd edition, p. 423-563. Edited by M. S. Balsam and E. Sagarin, Wiley Interscience, 1972.

The invention will now be described in further detail by way of the following examples.

### EXAMPLE 1

Differing flavour ingredients were added, at a concentration of 1000 ppm, to a non-flavoured toothpaste base containing 0.75% zinc citrate (ZCT).

Measured samples, of equal weight, of the non-flavoured toothpaste base containing 0.75% ZCT and the same toothpaste base modified with flavour ingredients were then placed on toothbrushes. The samples were tested by a panel of three independent trained experts. The panelists were instructed to brush their teeth for 30 seconds, spit out and rinse once with water. The subjects were then asked to rank the astringency on a scale of 0 to 10 for each sample, 0 representing no astringency and 10 representing strong astringency. A current market product was also tested in the same way.

Table 1 lists the flavour ingredients added to the non-flavoured toothpaste base containing 0.75% zinc citrate, along with the mean astringency rating of each sample. Also included in table 1 is the mean astringency rating of the non-flavoured toothpaste base containing 0.75% ZCT and the current market product.

As can be seen from the figures in table 1, the toothpaste compositions comprising the flavour materials of the invention, maltol ethyl maltol and cycoltene, all showed significantly lower astringency ratings compared to those of the standard toothpaste base and the current market product.

TABLE 1

| Ingredient* | Astringency Rating (0-10)¶ |
|---|---|
| Ethyl Maltol | 3.5 |
| Maltol | 3.8 |
| Cyclotene | 4.5 |
| Vanillin | 6.5 |
| Spearmint Oil | 7 |
| Orange Sweet | 7 |
| Non-flavoured Toothpaste base 0.75% ZCT | 8 |
| Current Market Toothpaste 0.75% ZCT | 6.5 |

*Ingredients tested at 1000 ppm (0.1% w/w) in Standard Toothpaste Base with 0.75% Zinc Citrate.

### EXAMPLE 2

Example compositions containing 2% Zinc Citrate with varying amounts of maltol, ethyl maltol and cyclotene are shown in Table 2.

TABLE 2
Example Composition

| FLAVOUR | | | | | |
|---|---|---|---|---|---|
| INGREDIENTS | 1 | 2 | 3 | 4 | 5 |
| ANETHOLE | 10.00 | 10.00 | 10.00 | 10.00 | 10.00 |
| CARVONE LAEVO | 3.00 | 3.00 | 3.00 | 3.00 | 3.00 |
| CYCLOTENE | 0.00 | 0.00 | 2.50 | 0.00 | 2.00 |
| ETHYL MALTOL | 1.00 | 0.00 | 0.00 | 0.50 | 0.50 |
| MALTOL | 0.00 | 1.00 | 0.00 | 0.50 | 0.00 |
| MENTHOL LAEVO | 35.00 | 35.00 | 35.00 | 35.00 | 35.00 |
| ORANGE SWEET | 0.00 | 0.00 | 0.00 | 0.00 | 0.00 |
| JAMAICAN | | | | | |
| PEPPERMINT OIL | 47.00 | 47.00 | 46.50 | 47.00 | 46.50 |
| BLEND | | | | | |
| PHENYL ETHYL | 0.00 | 0.00 | 0.00 | 0.00 | 0.00 |
| ALCOHOL | | | | | |
| PROPYLENE GLYCOL | 1.00 | 1.00 | 0.00 | 1.00 | 0.00 |
| SPEARMINT OIL | 3.00 | 3.00 | 3.00 | 3.00 | 3.00 |
| Total Quantity | 100.00 | 100.00 | 100.00 | 100.00 | 100.00 |
| % Astringency moderators | 1.00 | 1.00 | 2.50 | 1.00 | 2.50 |
| of the invention (based on | | | | | |
| flavour) | | | | | |
| PRODUCT | 1 | 2 | 3 | 4 | 5 |
| Base (2% Zinc Citrate | 98.75 | 98.75 | 98.75 | 98.75 | 98.75 |
| Toothpaste Base) | | | | | |
| Flavour | 1.00 | 1.00 | 1.00 | 1.00 | 1.00 |
| Saccharin | 0.25 | 0.25 | 0.25 | 0.25 | 0.25 |

Concentration of astringency moderators in
product (ppm by weight)

| | | | | | |
|---|---|---|---|---|---|
| Maltol | | 100.00 | | 50 | |
| Ethyl Maltol | 100.00 | | | 50 | 50 |
| Cycoltene | | | 250.00 | | 200 |
| Sum of M/50 + E/50 + | 2 | 2 | 1 | 2 | 1.8 |
| C/250 | | | | | |

Unless specified otherwise all concentrations are in percentage by weight based on the Total weight of the oral care composition

1. An oral care composition comprising at least one polyvalent metal ion salt and at least one of maltol (M), ethyl maltol (E), and or cyclotene (C) such that the concentration (in ppm by weight of the total oral care composition) of maltol (M), ethyl maltol (E), and cyclotene (C) meet the criterion (M/50+E/50+C/250)≧1.

2. An oral care composition according to claim 1 wherein the polyvalent metal ion salt is a zinc salt.

3. An oral care composition according to claim 2 wherein the zinc salt is zinc citrate.

4. The oral care composition according to claim 1 wherein, (M/50+E/50+C/250)=1.2 to 1.5.

5. The oral care composition according to claim 1 wherein the cyclotene together with maltol and/or ethyl maltol are employed in the oral care composition at a weight ratio 1:10-1:5 to 10:1-5:1.

6. (canceled)

7. (canceled)

8. A method of forming an oral care composition comprising the addition of at least one of maltol (M), ethyl maltol (E), or cyclotene (C) to said oral care composition such that the concentrations in the oral care composition, (in ppm by weight of the total oral care composition), of M, E and C meet the criterion (M/50+E/50+C/250)≧1.

9. A method according to claim 8 wherein, (M/50+E/50+C/250)=1.2−1.5.

10. A method according to claim 9 wherein the cyclotene together with maltol and/or ethyl maltol are employed in the oral care composition at a weight ratio 1:10-1:5 to 10:1-5:1.

11. The method according to claim 8 wherein the cyclotene is combined with one or both of maltol and/or ethyl maltol in a ratio of 1:10 to 10:1.

12. The method according to claim 8 wherein the cyclotene is combined with one or both of maltol and/or ethyl maltol in a ratio of 1:5 to 5:1.

13. The oral care composition according to claim 4 wherein the polyvalent metal ion salt is a zinc salt.

14. The oral care composition according to claim 4 wherein the zinc salt is zinc citrate.

15. The oral care composition according to claim 4 wherein the cyclotene is combined with one or both of maltol and/or ethyl maltol in a ratio of 1:10 to 10:1.

16. The oral care composition according to claim 15 wherein the polyvalent metal ion salt is a zinc salt.

17. The oral care composition according to claim 4 wherein the cyclotene is combined with one or both of maltol and/or ethyl maltol in a ratio of 1:5 to 5:1.
